SEQUENCE LISTING (45 sequences)

SEQ ID NO: 1 - DNA, 12 bp, Artificial sequence
  Barcode sequence 1
ctcgagtagcag

SEQ ID NO: 2 - DNA, 12 bp, Artificial sequence
  Barcode sequence 2
cagcacacgtca

SEQ ID NO: 3 - DNA, 12 bp, Artificial sequence
  Barcode sequence 3
acagcgatcgac

SEQ ID NO: 4 - DNA, 12 bp, Artificial sequence
  Barcode sequence 4
ctctctacagca

SEQ ID NO: 5 - DNA, 12 bp, Artificial sequence
  Barcode sequence 5
tagtcgtctagc

SEQ ID NO: 6 - DNA, 12 bp, Artificial sequence
  Barcode sequence 6
catcagctgcac

SEQ ID NO: 7 - DNA, 12 bp, Artificial sequence
  Barcode sequence 7
tagtagcacgca

SEQ ID NO: 8 - DNA, 12 bp, Artificial sequence
  Barcode sequence 8
cagtcagctgac

SEQ ID NO: 9 - DNA, 12 bp, Artificial sequence
  Barcode sequence 9
cagcagtctacg

SEQ ID NO: 10 - DNA, 12 bp, Artificial sequence
  Barcode sequence 10
cagctagagcac

SEQ ID NO: 11 - DNA, 12 bp, Artificial sequence
  Barcode sequence 11
acagcagcgtag

SEQ ID NO: 12 - DNA, 12 bp, Artificial sequence
  Barcode sequence 12
actctacgcgac

SEQ ID NO: 13 - DNA, 12 bp, Artificial sequence
  Barcode sequence 13
ctgtcgagctga

SEQ ID NO: 14 - DNA, 12 bp, artificial sequence
  Barcode sequence 14
acagacgagtca

SEQ ID NO: 15 - DNA, 12 bp, artificial sequence
  Barcode sequence 15
ctatgatctacg

SEQ ID NO: 16 - DNA, 12 bp, artificial sequence
  Barcode sequence 16
ctcagagcagac

SEQ ID NO: 17 - DNA, 12 bp, artificial sequence
  Barcode sequence 17
acagagactacg

SEQ ID NO: 18 - DNA, 12 bp, Artificial sequence
  Barcode sequence 18
ctctgcactagc

SEQ ID NO: 19 - DNA, 12 bp, Artificial sequence
  Barcode sequence 19
actagtgacgac

SEQ ID NO: 20 - DNA, 12 bp, Artificial sequence
  Barcode sequence 20
tacgatgcgtac

SEQ ID NO: 21 - DNA, 12 bp, Artificial sequence
  ACGAGACATCAC
acgagacatcac

SEQ ID NO: 22 - DNA, 12 bp, Artificial sequence
  Barcode sequence 22
catcactgcaca

SEQ ID NO: 23 - DNA, 82 bp, Artificial sequence
  oligodT 1
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatctctcgagtagcagnnnnnnnnnnnvvvvvttttttttttttttttttttttttttttttvn

SEQ ID NO: 24 - DNA, 82 bp, Artificial sequence
  oligodT 2
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatctcagcacacgtcannnnnnnnnnnvvvvvttttttttttttttttttttttttttttttvn

SEQ ID NO: 25 - DNA, 82 bp, Artificial sequence
  oligodT 3
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatctacagcgatcgacnnnnnnnnnnnvvvvvttttttttttttttttttttttttttttttvn

SEQ ID NO: 26 - DNA, 82 bp, Artificial sequence
  oligodT 4
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatctctctctacagcannnnnnnnnnnvvvvvttttttttttttttttttttttttttttttvn

SEQ ID NO: 27 - DNA, 82 bp, Artificial sequence
  oligodT 5
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatcttagtcgtctagcnnnnnnnnnnnvvvvvttttttttttttttttttttttttttttttvn

SEQ ID NO: 28 - DNA, 82 bp, Artificial sequence
  oligodT 6
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatctcatcagctgcacnnnnnnnnnnnvvvvvttttttttttttttttttttttttttttttvn

SEQ ID NO: 29 - DNA, 82 bp, Artificial sequence
  oligodT 7
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatcttagtagcacgcannnnnnnnnnnvvvvvttttttttttttttttttttttttttttttvn

SEQ ID NO: 30 - DNA, 82 bp, artificial sequence
  oligodT 8
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatctcagtcagctgacnnnnnnnnnnnvvvvvttttttttttttttttttttttttttttttvn

SEQ ID NO: 31 - DNA, 82 bp, artificial sequence
  oligodT 9
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatctcagcagtctacgnnnnnnnnnnnvvvvvttttttttttttttttttttttttttttttvn

SEQ ID NO: 32 - DNA, 82 bp, artificial sequence
  oligodT 10
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatctcagctagagcacnnnnnnnnnnnvvvvvttttttttttttttttttttttttttttttvn

SEQ ID NO: 33 - DNA, 82 bp, artificial sequence
  oligodT 11
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatctacagcagcgtagnnnnnnnnnnnvvvvvttttttttttttttttttttttttttttttvn

SEQ ID NO: 34 - DNA, 82 bp, artificial sequence
  oligodT 12
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatctactctacgcgacnnnnnnnnnnnvvvvvttttttttttttttttttttttttttttttvn

SEQ ID NO: 35 - DNA, 82 bp, artificial sequence
  oligodT 13
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatctctgtcgagctgannnnnnnnnnnvvvvvttttttttttttttttttttttttttttttvn

SEQ ID NO: 36 - DNA, 82 bp, artificial sequence
  oligodT 14
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatctacagacgagtcannnnnnnnnnnvvvvvttttttttttttttttttttttttttttttvn

SEQ ID NO: 37 - DNA, 82 bp, artificial sequence
  oligodT 15
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatctctatgatctacgnnnnnnnnnnnvvvvvttttttttttttttttttttttttttttttvn

SEQ ID NO: 38 - DNA, 82 bp, artificial sequence
  oligodT 16
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatctctcagagcagacnnnnnnnnnnnvvvvvttttttttttttttttttttttttttttttvn

SEQ ID NO: 39 - DNA, 82 bp, Artificial sequence
  oligodT 17
  misc_feature (35)..(45): wherein n stands for any nucleotide selected from A,C,G and T
  misc_feature (46)..(50): wherein v stands for any nucleotide selected from A,C and G
  misc_feature (81)..(81): wherein v stands for any nucleotide selected from A,C and G
  misc_feature (82)..(82): wherein v stands for any nucleotide selected from A,C and G
ctacacgacgctcttccgatctacagagactacgnnnnnnnnnnnvvvvvttttttttttttttttttttttttttttttvn

SEQ ID NO: 40 - DNA, 82 bp, Artificial sequence
  oligodT 18
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatctctctgcactagcnnnnnnnnnnnvvvvvttttttttttttttttttttttttttttttvn

SEQ ID NO: 41 - DNA, 82 bp, Artificial sequence
  oligodT 19
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatctactagtgacgacnnnnnnnnnnnvvvvvttttttttttttttttttttttttttttttvn

SEQ ID NO: 42 - DNA, 82 bp, Artificial sequence
  oligodT 20
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatcttacgatgcgtacnnnnnnnnnnnvvvvvttttttttttttttttttttttttttttttvn

SEQ ID NO: 43 - DNA, 82 bp, Artificial sequence
  CTACACGACGCTCTTCCGATCTACGAGACATCACNNNNNNNNNNNVV VVVTTTTTTTTTTTTTTTTTTTTTTTTTTTTTTVN
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatctacgagacatcacnnnnnnnnnnnvvvvvttttttttttttttttttttttttttttttvn

SEQ ID NO: 44 - DNA, 82 bp, Artificial sequence
  oligodT 22
  misc_feature (35)..(45): wherein n stands for any nucleotide selected from A,C,G and T
  misc_feature (46)..(50): wherein v stands for any nucleotide selected from A,C and G
  misc_feature (81)..(81): wherein  v stands for any nucleotide selected from A,C and G
  misc_feature (82)..(82): wherein n stands for any nucleotide selected from A,C,G and T
ctacacgacgctcttccgatctcatcactgcacannnnnnnnnnnvvvvvttttttttttttttttttttttttttttttvn

SEQ ID NO: 45 - DNA, 85 bp, Artificial sequence
  oligodT 23
  misc_feature (26)..(48): wherein n stands for any nucleotide selected from A,C,G and T
  misc_feature (49)..(53): wherein v stands for any nucleotide selected from A,C and G
  misc_feature (84)..(84): wherein  v stands for any nucleotide selected from A,C and G
  misc_feature (85)..(85): wherein n stands for any nucleotide selected from A,C,G and T
aagcagtggtatcaacgcagagtacnnnnnnnnnnnnnnnnnnnnnnnvvvvvttttttttttttttttttttttttttttttvn

(19) United States
(12) Patent Application Publication
(10) Pub. No.: US 2023/0366021 A1
(43) Pub. Date: Nov. 16, 2023

(54) METHOD OF PREPARATION OF cDNA LIBRARY USEFUL FOR EFFICIENT mRNA SEQUENCING AND USES THEREOF

(71) Applicant: ECOLE POLYTECHNIQUE FEDERALE DE LAUSANNE (EPFL), LAUSANNE (CH)

(72) Inventors: DANIEL ALPERN, EVIAN-LES-BAINS (FR); RICCARDO DAINESE, CHAVANNES-RENENS (CH); BART DEPLANCKE, CUGY (CH); MUSTAFA DEMIR, LAUSANNE (CH)

(21) Appl. No.: 18/029,113

(22) PCT Filed: Sep. 30, 2020
(86) PCT No.: PCT/EP2020/077437

§ 371 Date: Mar. 29, 2023

(51) Int. Cl. C12Q 1/6874; C12Q 1/6806

(52) U.S. Cl. CPC C12Q 1/6874; C12Q 1/6806; C12Q 2600/16

(57) ABSTRACT

The invention relates to methods for the preparation of method of preparation of cDNA library based on one or many RNA samples useful for efficient RNA sequencing and uses thereof. The invention further relates to related tools and kits useful in said method.

### FIELD OF THE INVENTION

The present invention pertains generally to the field of high-throughput sequencing methods and uses thereof and in particular methods of preparation of cDNA sequencing library for bulk mRNA sequencing.

### BACKGROUND OF THE INVENTION

High-throughput sequencing has become the method of choice for genome-wide transcriptomic analyses as its price has substantially decreased over the last years. Nevertheless, the high cost of standard RNA library preparation and the complexity of the underlying data analysis still prevent this approach from becoming as routine as quantitative PCR (qPCR), especially when many samples need to be analyzed. To alleviate this high cost, the emerging single-cell transcriptomics field implemented the sample barcoding/early multiplexing principle. This reduces both the RNA-seq cost and preparation time by allowing the generation of a single sequencing library that contains multiple distinct samples/cells (Ziegenhain et al., 2017, Mol. Cell 65, 631-643.e4). Such a strategy could also be of value to reduce the cost and processing time of bulk RNA sequencing of large sets of samples (Kilpinen et al., 2013, Science 342, 744-747; Waszak. et al., 2015, Cell 162, 1039-1050; Pradhan et al. 2017, Sci. Rep. 7, 42130). However, there have been surprisingly few efforts to explicitly adapt and validate the early-stage multiplexing protocols for reliable and affordable profiling of bulk RNA samples.

All RNA-seq library preparation methods are globally relying on the same molecular steps, such as reverse transcription (RT), fragmentation, indexing, and amplification. However, when compared side by side, one can observe variation in the order and refinement of these steps. Currently, the de facto standard workflow for bulk transcriptomics is the directional dUTP approach (Parkhomchuk et al. 2009, Nucleic Acids Res. 37, e123—e123; Levin et al., 2010, Nat. Methods 7, 709-715) and its commercial adaptation “Illumina TruSeq Stranded mRNA”. Both procedures evoke late multiplexing, which necessitates the processing of samples on a one-by-one basis. To overcome this limitation, the RNAtag-seq protocol implemented the barcoding of fragmented RNA samples, which allows for early multiplexing and generation of a sequencing library covering entire transcripts (Shishkin et al., 2015, Nat. Methods 12, 323-325). However, this protocol involves rRNA-depletion and bias-prone RNA adapter ligation (Fuchs et al., 2015, PLOS ONE 10, e0126049), which is relatively cumbersome and expensive. Although providing a significantly faster and cheaper alternative, other approaches such as QuantSeq (Lexogen) and LM-seq still require the user to handle every sample individually (Hou et al., 2015, Sci. Rep. 5, 9570).

In contrast, early multiplexing protocols designed for single-cell RNA profiling (CEL-seq2, SCRB-seq, and STRT-seq) provide a great capacity for transforming large sets of samples into a unique sequencing library (Hashimshony et al. 2016, Genome Biol., 17, 77; Islam et al., 2012, Nat. Protoc. 7, 813-828; Soumillonet al., 2014, bioRxiv, 003236, doi:10.1101/003236). This is achieved by introducing a sample-specific barcode during the RT reaction using a 6-8 nt tag carried by either the oligo-dT or the template switch oligo (TSO). After individual samples have been labeled, they are pooled together, and the remaining steps are performed in bulk, thus shortening the time and cost of library preparation. Since the label is introduced to the terminal part of the transcript prior to fragmentation, the reads solely cover the 3′ or 5′ end of the transcripts. Therefore, the principal limitation of this group of methods is the incapacity to address splicing, fusion genes, or RNA editing-related research questions. However, most transcriptomics studies do not require or exploit full transcript information, implying that standard RNA-seq methods tend to generate more information than is typically required. This unnecessarily inflates the overall experimental cost, rationalizing why 3′-end profiling approaches such as the 3′ digital gene expression (3′DGE) assay have already been proven effective to determine genome-wide gene expression levels, although with a slightly lower sensitivity than conventional mRNA-seq (Xiong et al., 2017, Sci. Rep. 7, 14626).

The 3′DGE approach for bulk RNA profiling, has been adopted in several recent studies, such as PLATE-seq (Bush et al., 2017, Nat. Commun. 8, 105), DRUG-seq (Ye et al., 2018, Nat. Commun. 9, 1-9), 3′POOL-seq (Sholder et al., 2020, BMC Genomics 21, 64), PME-seq (Pandey et al., 2020, Nat. Protoc., 15, 1459-1483) and BRB-seq (Alpern et al., 2019 Genome Biol. 20, 71). These techniques have two main commonalities: 1) using barcoded DNA oligos used to “tag” poly-adenylated RNA molecules during first strand synthesis and ii) pooling together of all the tagged samples in one tube after the barcoding step.

The overarching goal of these techniques is to decrease the costs and increase the throughput associated with mRNA sequencing library preparation of bulk samples. This is achieved by reducing reagents, consumables and personnel time through pooling in one solution several barcoded samples. In simple terms, it is much more cost-effective and simpler to process e.g. 100 samples in one tube than 100 samples in 100 tubes. The methods that use barcoded DNA oligos for bulk transcriptomics, i.e. all the methods for high-throughput transcriptomics mentioned above, i.e. PLATE-seq, DRUG-seq, 3′POOL-seq, PME-seq and BRB-seq do not address the problem of RNA normalization before pooling. Moreover, the extraction step (even if performed with beads, as in PME-seq and 3′POOL-seq) is performed in a separate reaction before the barcoding step. Methods that use of non-magnetic beads coupled with barcoded DNA oligos such as Drop-seq (Macosko et al., 2015, Cell 161, 1202-1214) and inDrop (Klein et al., 2015, Cell 161, 1187-1201) use of beads coupled to barcoded oligos for single cell RNA-seq. In this case, each bead has a different barcode because each bead needs to capture the mRNA of only one cell. The beads are therefore used for mRNA capturing but do not allow any normalizing of the amount of captured mRNA. Moreover, these beads are not magnetic and do not rely on the streptavidin-biotin interaction, which is an important factor to ensure normalization of bulk samples.

Regarding the main differences among these approaches, they mostly lie in the steps that follow pooling the samples in one tube. Given the absence of a systematic comparison of these different strategies, it remains unclear whether there is a single winner in terms of quality, costs and throughput. If anything, due to the minor differences in the post-pooling strategies, all methods are practically equivalent. This is because the changes in performance due to different post-pooling strategies are overshadowed by the importance of the barcoding and pooling steps.

Therefore, barcoding and pooling many RNA samples in one tube leads to a significantly higher downstream efficiency in resources utilization. As such, this is the upstream step—and the reagents/consumables used therein—which become the main limiting factors in terms of experimental performance, i.e. cost and throughput. These steps are 1) RNA extraction, ii) RNA quantity normalization across samples and iii) the actual barcoding step.

Each of these steps involves a significant experimental effort:
i) RNA extraction is the process by which RNA molecules are purified from complex biological mixtures, typically cell lysates. This has to be performed individually for each sample and usually requires dedicated commercial kits or reagents. Approximately, this requires 500 CHF in consumables and one day of manual labor for 100 samples; ii) RNA quantity normalization ensures that the amount of RNA in the samples is uniform before pooling. This is a very important step for any method involving early sample pooling because the quantity of RNA in different samples can vary significantly. If this variability is not removed before pooling, it will translate in a sample pool containing different amounts of different samples. Downstream, this leads to variability in the number of sequencing reads that each sample will obtain after next generation sequencing. For example, if one sample is 10× more concentrated than another one, it will obtain 10× more sequencing reads. This can result in a substantial technical bias and the sample obtaining significantly less reads may need to be removed from the analysis and re-sequenced, which therefore greatly increases experimental costs. Therefore, a uniform sample quantity distribution before pooling ensures a uniform amount of sequencing reads across samples and maximizes experimental efficiency. This is why the normalization step is crucial, despite its cumbersome workflow, i.e. the concentration of each samples needs to be measured and the volume of each sample needs to be manually adjusted. This procedure leads per se to higher experimental costs, which increase proportionally with the number of input samples. Typically for 100 samples, this step requires an extra half day of manual work; ii) RNA barcoding is the last essential step before pooling and it requires two main elements: a) barcoded DNA oligos-dT (or TSO, template switch oligo) and b) the reverse transcriptase (RT) enzyme. Briefly, the barcoded oligos-dT contain a known variable sequence of nucleotides (barcode) and a stretch of 25-30 dT nucleotides, which enables it to anneal to the poly-dA tail of mRNA molecules. Oligo-dT serves as primer for RNA dependent DNA polymerase, capable of synthetizing first cDNA strand on the RNA template. The resulting cDNA will therefore contain the barcode sequence at its 5′ terminal. Since the barcode is sample specific all the cDNA molecules will contain the same barcode, allowing subsequently to pool the samples together. This process usually takes half-day of manual work and incubation.

Therefore, the development of new methods of high-throughput sequencing with efficient pre-pooling steps would be desirable to reduce experimental time and costs that are the limiting factors for further development of testing output in this field.

### SUMMARY OF THE INVENTION

The present invention is based on the unexpected finding that it is possible to integrate the three main pre-pooling steps before RNA sequencing on bulk samples in one single reaction step, thereby drastically reducing the experimental complexity, efforts and costs for the pre-pooling steps and thereby fully benefiting of the advantages of sample pooling strategies in high-throughput sequencing. The method of the invention is based on the use, before RNA sample pooling, of an internal RNA normalization tool specific for each RNA sample allowing ponderation of the contribution of each sample from the RNA pool in the sequencing read-out. This method can be advantageously used for any RNA sample (bulk or single cell).

A general object of this invention is to provide a method of preparing a cDNA library from pooled RNA samples, said library being useful for efficient bulk RNA sequencing.

One of the specific objects of this invention is to provide a method of preparing cDNA library of pooled bulk RNA samples wherein the quantity of each bulk sample within the library is controlled and normalized.

It is advantageous to provide a method of preparation of a cDNA library of pooled bulk samples wherein the amount of cDNA from each sample present in the pool is essentially identical to circumvent unequal distribution or reads of each sample according the library.

It is advantageous to provide a method of preparation of a cDNA library of pooled bulk RNA samples suitable for high-throughput accurate RNA sequencing.

Another of the specific objects of this invention is to provide a method of bulk RNA sequencing that is cost effective and accurate.

Objects of this invention have been achieved by providing a method for the preparation of cDNA library according to claim 1 useful for high-throughput sequencing.

Objects of this invention have been achieved by providing a method of bulk RNA sequencing according to claim 10.

Objects of this invention have been achieved by providing a kit according to claim 13.

Disclosed herein is a method for the preparation of a cDNA library based on several bulk mRNA samples comprising the steps of:
i) Providing separately a plurality of mRNA samples;
ii) Contacting separately each mRNA sample with biotinylated and barcoded oligo-dT sequences wherein the biotinylated and barcoded oligo-dT sequences are biotinylated at their 5′ end under annealing conditions to obtain, for each sample, sample-specific barcoded mRNA complexes (comprising a sample-specific barcoded oligo-dT primer bound to sample mRNA molecules);
iii) Contacting separately for each sample, said sample-specific barcoded mRNA complexes with streptavidin magnetic beads at a pre-defined concentration, said pre-defined concentration being identical for all mRNA samples;
iv) Incubating separately each sample with reverse transcription enzyme (RT) under reverse transcription reaction conditions;
v) Isolating separately for each sample the magnetic beads from the reaction medium;
vi) Pooling together in a single set of samples all the isolated magnetic beads from each sample to obtain a cDNA library.

Also disclosed herein is a method of bulk RNA sequencing, said method comprising the steps of:
providing a cDNA library comprising a plurality of sample-specific barcoded cDNAs, wherein said sample-specific barcoded cDNAs correspond to a unique bulk mRNA sample defined by its unique barcode and wherein the contribution of each sample in the cDNA library is the same;
amplifying said cDNAs from said library;
sequencing the amplification products.

Also disclosed herein is a cDNA library comprising a plurality of sample-specific barcoded cDNAs, wherein said sample-specific barcoded cDNAs correspond a unique mRNA sample defined by its unique barcode, wherein the contribution of each sample in the cDNA library is the same. According to one aspect, said cDNA library being useful for bulk RNA sequencing.

Disclosed herein is a kit useful for RNA sequencing, said kit comprising biotinylated and barcoded oligo-dT primers according to the invention and strepavidin magnetic beads or magnetic beads, wherein said strepavidin magnetic beads are optionally pre-functionalized with said barcoded DNA.

Other features and advantages of the invention will be apparent from the claims, detailed description, and figures.

### BRIEF DESCRIPTION OF THE DRAWINGS

FIG. 1 describes the main steps of a method of the invention for the preparation of a cDNA library based on a plurality of mRNA samples S1-S3 wherein in said cDNA library (SP), the contribution of each mRNA is the same due to the normalization achieved by the method of the invention.

FIG. 2 shows RT-qPCR quantification of RNA captured by variable amount of streptavidin beads as described in Example 1.

FIG. 3 shows the fold change difference between the total number of sequencing reads as “unique sequence identifies” (UMIs) of each sample in individual library with varying amount of oligo-dT primer (A) and after beads normalization (B) measures as describe in Example 2.

### DETAILED DESCRIPTION OF EMBODIMENTS OF THE INVENTION

The expression “bulk” when applied to RNA, refers to multiple cells as opposed to single cell. For example, in bulk sequencing, the measured data points do not correspond to single cells, but rather represent bulk samples (many cells).

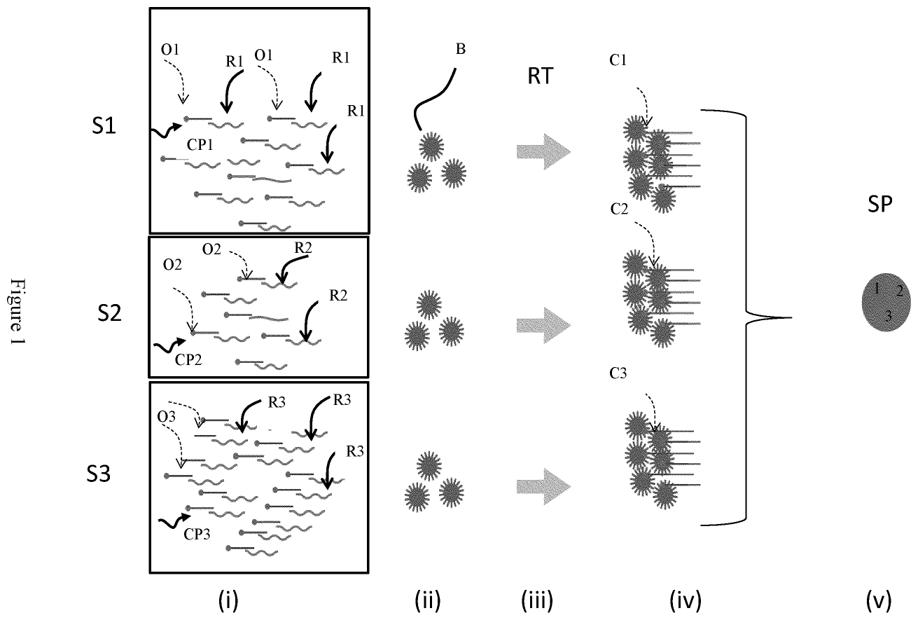

Referring to the figures, in particular first to FIG. 1, is provided an illustration of a method for the preparation of cDNA library based on several mRNA samples according to an embodiment of the invention. The illustrated method generally comprises the steps of:
i) Providing separately a plurality of mRNA samples S1-S3;
ii) Contacting separately each mRNA sample containing the mRNA material (R1, R2 or R3 respectively) with biotinylated and barcoded oligo-dT sequences (O1, O2 or O3, respectively) wherein the biotinylated and barcoded oligo-dT sequences are biotinylated at their 5′ end under annealing conditions to obtain, for each sample, a mixture comprising sample-specific barcoded mRNA complexes, CP1, CP2, CP3, respectively (comprising a sample-specific barcoded oligo-dT primer bound to sample mRNA molecules);
iii) Contacting separately for each sample, the obtained mixture with streptavidin magnetic beads (B) at a pre-defined concentration, said pre-defined concentration being identical for all mRNA samples;
iv) Incubating separately each sample in presence of a reverse transcription enzyme (RT) under reverse transcription reaction conditions;
v) after completion of the reverse transcription reaction, isolating the magnetic beads from the reaction medium for each sample;
vi) pooling together in a single set of samples all the isolated magnetic beads from each sample to obtain a cDNA library.

According to a particular aspect, the mRNA samples can be cell lysates or total DNA/RNA eluate. Those can be obtained by standard methods known to the skilled person.

According to a particular aspect, the reverse transcription enzyme which is used to transfer the elongated DNA olignucleotides and copy onto them the sequence of the captured RNA molecule.

According to a particular aspect, biotinylated and barcoded oligo-dT sequences comprise each:
a known sequence specific for each sample (barcode sequence);
a single strand sequence of deoxy-thymidine (dT) which is capable to anneal to any poly-A tail of mRNA molecules;
a biotin group modification of 5′ end of the oligo-dT primer.

According to a further particular embodiment, a sequence useful as barcode sequence can be of 6 to about 20 nucleotide long. Examples of those sequences comprise or consist in the following sequences:

(SEQ ID NO: 1)
CTCGAGTAGCAG;
(SEQ ID NO: 2)
CAGCACACGTCA;
(SEQ ID NO: 3)
ACAGCGATCGAC;
(SEQ ID NO: 4)
CTCTCTACAGCA;
(SEQ ID NO: 5)
TAGTCGTCTAGC;
(SEQ ID NO: 6)
CATCAGCTGCAC;
(SEQ ID NO: 7)
TAGTAGCACGCA;
(SEQ ID NO: 8)
CAGTCAGCTGAC;
(SEQ ID NO: 9)
CAGCAGTCTACG;
(SEQ ID NO: 10)
CAGCTAGAGCAC;
(SEQ ID NO: 11)
ACAGCAGCGTAG;
(SEQ ID NO: 12)
ACTCTACGCGAC;
(SEQ ID NO: 13)
CTGTCGAGCTGA;
(SEQ ID NO: 14)
ACAGACGAGTCA;
(SEQ ID NO: 15)
CTATGATCTACG;
(SEQ ID NO: 16)
CTCAGAGCAGAC;
(SEQ ID NO: 17)
ACAGAGACTACG;
(SEQ ID NO: 18)
CTCTGCACTAGC;
(SEQ ID NO: 19)
ACTAGTGACGAC;
(SEQ ID NO: 20)
TACGATGCGTAC;
(SEQ ID NO: 21)
ACGAGACATCAC;
(SEQ ID NO: 22)
CATCACTGCACA

or fragment thereof.

According to a particular aspect, biotinylated and barcoded oligo-dT sequences according to the invention are used for priming the reaction catalyzed with RT.

According to a particular aspect, a single strand sequence of deoxy-thymidine (dT) useful in a method of the invention targets any polyadenylated transcript present in the sample.

According to a particular aspect, the oligo-dT sequences comprises a single strand sequence of deoxy-thymidine (dT) of 2 to about 200 nucleotide long. Examples of single strand sequence of deoxy-thymidine (oligo dT) useful in a method of the invention comprise or consist in the following sequences:

(SEQ ID NO: 23)
CTACACGACGCTCTTCCGATCTCTCGAGTAGCAGNNNNNNNNNNNVVVVVTTTTTTT
TTTTTTTTTTTTTTTTTTTTTTTVN;
(SEQ ID NO: 24)
CTACACGACGCTCTTCCGATCTCAGCACACGTCANNNNNNNNNNNVVVVVTTTTTTT
TTTTTTTTTTTTTTTTTTTTTTTVN;
(SEQ ID NO: 25)
CTACACGACGCTCTTCCGATCTACAGCGATCGACNNNNNNNNNNNVVVVVTTTTTTT
TTTTTTTTTTTTTTTTTTTTTTTVN;
(SEQ ID NO: 26)
CTACACGACGCTCTTCCGATCTCTCTCTACAGCANNNNNNNNNNNVVVVVTTTTTTTT
TTTTTTTTTTTTTTTTTTTTTTVN;
(SEQ ID NO: 27)
CTACACGACGCTCTTCCGATCTTAGTCGTCTAGCNNNNNNNNNNNVVVVVTTTTTTTT
TTTTTTTTTTTTTTTTTTTTTTVN;
(SEQ ID NO: 28)
CTACACGACGCTCTTCCGATCTCATCAGCTGCACNNNNNNNNNNNVVVVVTTTTTTT
TTTTTTTTTTTTTTTTTTTTTTTVN;
(SEQ ID NO: 29)
CTACACGACGCTCTTCCGATCTTAGTAGCACGCANNNNNNNNNNNVVVVVTTTTTTT
TTTTTTTTTTTTTTTTTTTTTTTVN;
(SEQ ID NO: 30)
CTACACGACGCTCTTCCGATCTCAGTCAGCTGACNNNNNNNNNNNVVVVVTTTTTTT
TTTTTTTTTTTTTTTTTTTTTTTVN;
(SEQ ID NO: 31)
CTACACGACGCTCTTCCGATCTCAGCAGTCTACGNNNNNNNNNNNVVVVVTTTTTTT
TTTTTTTTTTTTTTTTTTTTTTTVN;
(SEQ ID NO: 32)
CTACACGACGCTCTTCCGATCTCAGCTAGAGCACNNNNNNNNNNNVVVVVTTTTTTT
TTTTTTTTTTTTTTTTTTTTTTTVN;
(SEQ ID NO: 33)
CTACACGACGCTCTTCCGATCTACAGCAGCGTAGNNNNNNNNNNNVVVVVTTTTTTT
TTTTTTTTTTTTTTTTTTTTTTTVN;
(SEQ ID NO: 34)
CTACACGACGCTCTTCCGATCTACTCTACGCGACNNNNNNNNNNNVVVVVTTTTTTT
TTTTTTTTTTTTTTTTTTTTTTTVN;
(SEQ ID NO: 35)
CTACACGACGCTCTTCCGATCTCTGTCGAGCTGANNNNNNNNNNNVVVVVTTTTTTT
TTTTTTTTTTTTTTTTTTTTTTTVN;
(SEQ ID NO: 36)
CTACACGACGCTCTTCCGATCTACAGACGAGTCANNNNNNNNNNNVVVVVTTTTTTT
TTTTTTTTTTTTTTTTTTTTTTTVN;
(SEQ ID NO: 37)
CTACACGACGCTCTTCCGATCTCTATGATCTACGNNNNNNNNNNNVVVVVTTTTTTTT
TTTTTTTTTTTTTTTTTTTTTTVN;
(SEQ ID NO: 38)
CTACACGACGCTCTTCCGATCTCTCAGAGCAGACNNNNNNNNNNNVVVVVTTTTTTT
TTTTTTTTTTTTTTTTTTTTTTTVN;
(SEQ ID NO: 39)
CTACACGACGCTCTTCCGATCTACAGAGACTACGNNNNNNNNNNNVVVVVTTTTTTT
TTTTTTTTTTTTTTTTTTTTTTTVN;
(SEQ ID NO: 40)
CTACACGACGCTCTTCCGATCTCTCTGCACTAGCNNNNNNNNNNNVVVVVTTTTTTTT
TTTTTTTTTTTTTTTTTTTTTTVN;
(SEQ ID NO: 41)
CTACACGACGCTCTTCCGATCTACTAGTGACGACNNNNNNNNNNNVVVVVTTTTTTT
TTTTTTTTTTTTTTTTTTTTTTTVN;
(SEQ ID NO: 42)
CTACACGACGCTCTTCCGATCTTACGATGCGTACNNNNNNNNNNNVVVVVTTTTTTT
TTTTTTTTTTTTTTTTTTTTTTTVN;
(SEQ ID NO: 43)
CTACACGACGCTCTTCCGATCTACGAGACATCACNNNNNNNNNNNVVVVVTTTTTTT
TTTTTTTTTTTTTTTTTTTTTTTVN;
(SEQ ID NO: 44)
CTACACGACGCTCTTCCGATCTCATCACTGCACANNNNNNNNNNNVVVVVTTTTTTT
TTTTTTTTTTTTTTTTTTTTTTTVN
and
(SEQ ID NO: 45)
AAGCAGTGGTATCAACGCAGAGTACNNNNNNNNNNNNNNNNNNNNNNNVVVVVTT
TTTTTTTTTTTTTTTTTTTTTTTTTTTTVN,

wherein V stands for any nucleotide selected from A,C and G and N stands for any nucleotide selected from A,C,G and T.

According to a particular aspect, step ii) is conducted under annealing conditions. Example of typical annealing conditions useful in a method of the invention are described in Newbold et al., 2014, Cold Spring Harb Protoc; doi:10.1101/pdb.prot08253 7.

According to a particular embodiment, for each mRNA sample steps i) to iv) can be conducted sequentially or at once in a single step.

According to a particular aspect, the mRNA material is contacted with strepavidin magnetic beads or magnetic beads pre-functionalized with barcoded oligo-dT sequences.

According to a particular embodiment, step iv) is typically conducted for about 30 minutes to about 4 hours. Example of typical reverse transcription reaction conditions useful in a method of the invention are described in Newbold et al., 2014, Cold Spring Harb Protoc; doi: 10.1101/pdb.prot08253 7.

According to a particular aspect, in each sample, magnetic beads are carrying sample-specific barcoded cDNAs wherein said sample-specific barcoded cDNAs are barcoded on its 5′ terminal end with a sequence which is specific to the corresponding mRNA sample.

According to a particular embodiment, isolation of the magnetic beads is carried out by magnetic force such as by placing the well-plate containing the samples in a dedicated and commercially available magnetic plate holder.

In a particular aspect, biotinylated and barcoded DNA oligonucleotides, which will capture RNA molecules by their poly-A tail and will be captured at the same time by the streptaviding magnetic beads.

According to a particular aspect, in each sample, the mRNA molecules will anneal to the oligo-dT single strand sequence of deoxy-thymidine (dT) (primers) containing a sample-specific barcode and the fact that the barcoded primers are biotinylated allows them to strongly bind to the streptavidin magnetic beads, which advantageously makes extremely easy to purify the reverse transcription products and instead of using dedicated extraction kits, the operator can simply extract the beads by magnetic force. The first strand synthesis reaction under step iv) can advantageously be performed directly on the beads with mRNA molecules immobilized.

According to another, is provided a method of bulk RNA sequencing, said method comprising the steps of:
providing a cDNA library comprising a plurality of sample-specific barcoded cDNAs, wherein said sample-specific barcoded cDNAs correspond to a unique bulk mRNA sample defined by its unique barcode and wherein the contribution of each sample in the cDNA library is the same;
amplifying said cDNAs from said library;
sequencing the amplification products.

According to a particular aspect, the amplifying of cDNAs from a library prepared according to the invention comprises standard steps such as fragmentation or tagmentation, adapter ligation, DNA amplification, concentration measurement and DNA size distribution profiling in order to proceed with the sequencing of the amplified products,

The fact that the magnetic beads are introduced at defined amounts allows for normalization to occur within the same reaction. According to a particular aspect, the amount of magnetic beads which is added to each sample is such so that the quantity of complexes formed by magnetic beads carrying sample-specific barcoded cDNAs that is purified by magnetic separation cannot exceed a pre-determined amount. This amount is equal to the overall binding capacity of the added beads and it provides an effective cut-off to the amount of RNA that each sample brings to the pool.

According to a particular aspect, the cDNAs attached to the beads can advantageously be amplified using standard molecular biology practices, i.e. second strand synthesis and PCR amplification, be sequenced using next generation sequencing machines.

According to a particular aspect, is provided a kit comprising:
biotinylated and barcoded oligo-dT primers according to the invention and
strepavidin magnetic beads or magnetic beads,
   wherein said strepavidin magnetic beads are optionally pre-functionalized with said barcoded DNA.

According to a further particular aspect, a kit of the invention may further comprise at least one of the following elements:
a reverse transcription enzyme;
buffers to carry out the CDNAs normalisation and/or reverse transcription reactions.

According to a further particular aspect, is provided a kit of the invention, wherein said deoxy-thymidine (oligo dT) sequences are selected from the sequences from SEQ ID NO: 23 to SEQ ID NO: 45.

According to a particular aspect, a kit according to the invention is useful for sample preparation for RNA sequencing. More specifically, a kit according to the invention allows treating an arbitrary number of RNA samples and to generate one cDNA pool for further sequencing, said pool being characterized by a uniform representation of each sample in the pool.

According to an advantageous aspect of the invention, the strepavidin magnetic beads will serve as the solid state substrate for RNA capture and importantly, the main normalising agents for the generation of the CDNA pool.

According to an advantageous aspect of the invention, the pre-defined and uniform distribution of the magnetic beads before the pooling of various samples ensures that the distribution of sequencing reads for each sample does not exceed a predefined amount. This, in turn, avoids the typical unwanted situation in which few samples collected the majority of the sequencing reads, while many samples are left with too few reads. This situation is particularly unwanted because the samples with few reads need to be re-sequenced, which in turn greatly increases overall costs.

Further, since with bulk samples, the use of barcoded beads to extract and barcode bulk samples and at the same time to normalize their quantity in the pool according to a method of the invention provides a clearly advantageous, unique and innovative method for RNA sequencing of bulk samples.

The invention having been described, the following examples are presented by way of illustration, and not limitation.

### EXAMPLES

### Example 1: Normalization of Samples in the Library with Variable Input RNA Amounts

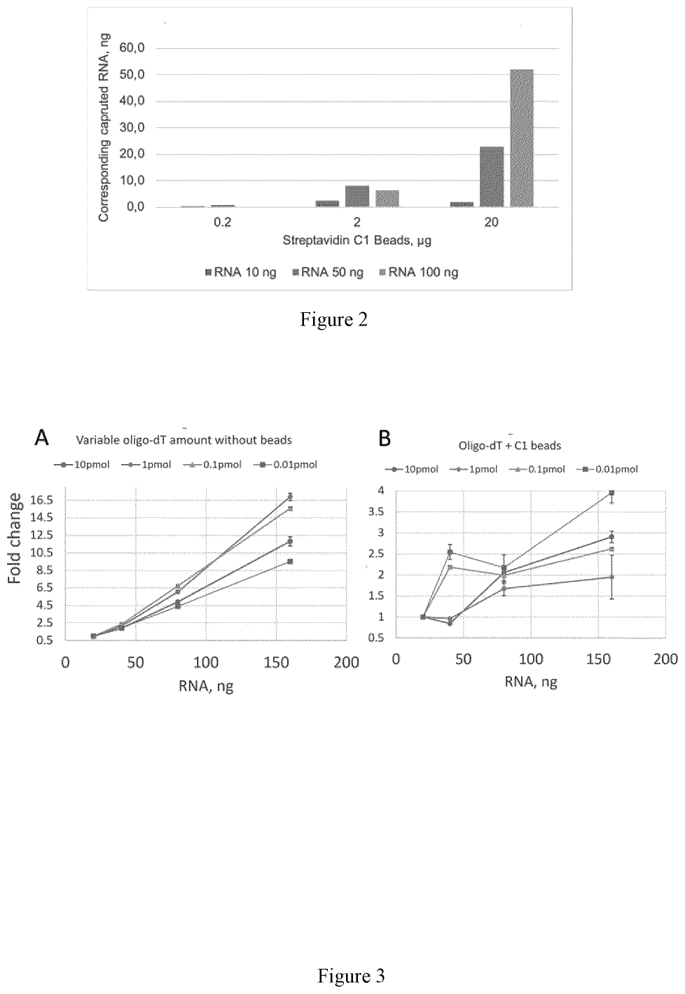

To test whether it is possible to cut-off on the maximum amount of RNA molecules to be pooled together from each sample, the RT reaction was tested using variable initial amount of RNA (20, 40, 80, 160 ng/well) and pulled down the resulting cDNA with variable amount of C1 beads (0.2, 2, 20 μg) (Step i) of the method of the invention). Aliquots were taken to assess the relative amount of captured RNA by qPCR and the rest was used for BRB-seq libraries preparation on the beads. FIG. 2A shows that overall amount of captured RNA is proportional to the quantity of used C1 beads. With 20 μg, the differences between each RNA is proportional to the input amount. However, with 2 μg of beads the captured amount of RNA is very similar between the samples with initial input of 50 and 100 ng.

Next, the sequencing libraries were prepared using variable amounts RNA (20, 40, 80, 160 ng/well) and variable quantity of either oligo-dT primer (BU3V3, 5′-biotin-labelled AAGCAGTGGTATCAACGCAGAGTAC VVVVVTT TTTTTTTTTTTTTTTTTTTTTTTTTTTTVN) (St NO: 45) (0.01, 0.1, 1, 10 pmol) (FIG. 3A, comparative method) or C1 beads (0.2, 2, 20 μg) (FIG. 3B method of the invention).

This experiment demonstrates that variable oligo-dT amount cannot obliterate the differences in the read distribution in the library across samples with varying input quantity. However, when cDNA is captured with the C1 beads the samples with the high input amount (40-160 ng) obtain very similar number of sequencing reads and therefore their proportions in the pools captured with all tested bead amounts are very close. Together this provides the evidence that RNA libraries can be normalized by using predefined amount of C 1 beads in order to bypass uneven distribution of sequencing reads caused by variable input RNA amounts per well.

### Modified BRB-Seq Protocol (Comparative Example, i.e. Without Beads)

### Step a) RNA Reverse Transcription as in Original BRB-Seq Protocol

96 RNA samples from HEK293 cells were reverse transcribed in a 96-well plate using Maxima H Minus Reverse Transcriptase (MMH, ThermoFisher Scientific, #EP0753) with individual biotinylated and barcoded oligo-dT primers (SEQ ID NO: 45), where first 10-Ns represent the barcode, and next 13-Ns +5-Vs is a UMI; IDT, Belgium).

### Step b) Library Preparation as in Standard BRB-Seq: Pooling, Purification, Exonuclease Digestion, Second Strand Synthesis.

Next, the samples were pooled corresponding to each library, purified using the DNA Clean and Concentrator kit (Zymo Research #D4014) and eluted in 20 μL of water. Residual primers were digested by adding 1 μL of Exonuclease I (NEB or New England BioLabs #M0293S) and 2 μL of 10× ExoI buffer (NEB) for 30 minutes at 37° C. followed by inactivation during 20 minutes at 80° C. Double-stranded cDNA was generated via the second stand synthesis by adding 1 μL of RNAse H (NEB, #M0297S), 1 μL of Bst2.0 WarmStart DNA Polymerase (NEB, #M0538S), 2.5 μL of 10× isothermal buffer (NEB) and 2 μL of 10 mM dNTP mix (ThermoFisher, #R0192) added to 20 μL of ExoI-treated first-strand reaction on ice. The reaction was incubated at 37° C. for 20 minutes followed by 65° C. for 30 minutes. 25 μL of water was added to the final volume of 50 μL and full-length double-stranded cDNA was purified with 30 μL (0.6×) of AMPure XP magnetic beads (Beckman Coulter, #A63881) and eluted in 20 μL of water.

### Step c) Illumina-Compatible Library Preparation (Tagmentation, Purification, Amplification and Size Selection)

The Illumina compatible libraries were prepared by tagmentation of 5 ng of full-length double-stranded cDNA with 1 μL of in-house produced Tn5 enzyme (11 μM). After tagmentation the libraries where purified with DNA Clean and Concentrator kit (Zymo Research #D4014), eluted in 20 μL of water and PCR amplified using 25 μL NEBNext High-Fidelity 2× PCR Master Mix (NEB, #M0541 L), 2.5 μL of P5_BRB primer (5 μM, Microsynth), and 2.5 μL of Illumina index adapter (Idx7N5 5 μM, IDT) following program: incubation 72° C. for 3 min, denaturation 98° C. for 30 s; 15 cycles: 98° C.-10 s, 63° C. for 30 s, 72° C. for 30 s; final elongation at 72° C. for 5 min. The fragments ranging 200-1000 bp were size-selected using AMPure beads (Beckman Coulter, #A63881) (first round 0.5× beads, second 0.7×).

### Step d) Final QC and Illumina Sequencing

The libraries were profiled with High Sensitivity NGS Fragment Analysis Kit (Advanced Analytical, #DNF-474) and measured with Qubit dsDNA HS Assay Kit (Invitrogen, #Q32851) prior to pooling and sequencing using the Illumina NextSeq 500 platform using a custom primer and the High Output v2 kit (75 cycles) (Illumina, #FC-404-2005). The library loading concentration was 2.2 pM and sequencing configuration as following: Read1 21 cycles/index read 8 cycles/Read2 62 cycles.

### cDNA Normalization with Beads According to the Method of the Invention

### Step i) Reverse Transcription (Same as in Comparative Example Step a)

Variable amount of RNA (20, 40, 80 or 160 ng/well) each in 3 replicates was transferred into each of 8 rows of 96 well plate and used for the first strand synthesis following the standard BRB-seq protocol.

Steps ii)-vi) Bead-Based Normalization and Pooling

After that variable amount of (0.2, 2, 20 μg) pre-washed streptavidin coated paramagnetic beads (Dynabeads C1, Thermo Fischer) were transferred into the rows of plate in duplicate. The plate was incubated in the shaker (1′000 rpm) at room temperature for 15 minutes. After that, the beads were washed twice with WB to remove unbound cDNA and the wells in each row were pooled together.

The library was then prepared as in standard BRB-seq (Steps a), b) and c) of the comparative example above (preparation and amplifying cDNAs in view of sequencing, as described in the present application).

### Pre-Processing of the Data—Demultiplexing and Alignment

The sample reads demultiplexing was done using BRB-seqTools (http://github.com/DeplanckeLab/BRB-segTools) as described before (Alpern et al. 2019, Genome Biol., 20, 71).). The sequencing reads were aligned to the Ensembl gene annotation of the homo sapience GRCh38.100.100 genome using STAR (version 020201) (Dobin et al. 2013, Bioinforma. Oxf. Engl., 29, 15-21).), and count matrices were generated with HTSeq (version 0.9.1) (Love et al., 2014, Genome Biol., 15, 550). The demultiplexed gene count data was further analyzed using R software.

1-15. (canceled)

16. A method for the preparation of a cDNA library based many RNA samples comprising the steps of:
i) providing separately a plurality of mRNA samples;
ii) contacting separately each mRNA sample with biotinylated and barcoded oligo-dT sequences wherein the biotinylated and barcoded oligo-dT sequences are biotinylated at their 5′ end under annealing conditions to obtain, for each sample, sample-specific barcoded mRNA complexes;
iii) contacting separately for each sample, said sample-specific barcoded mRNA complexes with streptavidin Magnetic Beads at a pre-defined concentration, said pre-defined concentration being identical for all mRNA samples;
iv) incubating separately each sample with reverse transcription enzyme (RT) under reverse transcription reaction conditions;
v) isolating separately for each sample the magnetic beads from the reaction medium; and
vi) pooling together in a single set of samples all the isolated magnetic beads from each sample to obtain a cDNA library.

17. The method according to claim 16, wherein steps i) to iv) are conducted at once in a single step.

18. The method according to claim 17, wherein the mRNA material is contacted with strepavidin magnetic beads or magnetic beads pre-functionalized with barcoded oligo-dT sequences.

19. The method according to claim 16, wherein biotinylated and barcoded oligo-dT sequences comprise each: a known sequence specific for each sample (barcode sequence); a single strand sequence of deoxy-thymidine (dT) which is capable to anneal to any poly-A tail of mRNA molecules; and a biotin group.

20. The method according to claim 16, wherein barcoded oligo-dT sequences comprises a barcode sequence are 6 to about 20 nucleotide long.

21. The method according to claim 16, wherein the oligo-dT sequences comprises a single strand sequence of deoxy-thymidine (dT) 2 to about 200 nucleotide long.

22. The method according to claim 16, wherein step iv) is conducted for about 30 minutes to about 4 hours.

23. The method according to claim 16, wherein in each sample magnetic beads are carrying sample-specific barcoded cDNAs wherein said sample-specific barcoded cDNAs are barcoded on its 5′ terminal end with a sequence which is specific to the corresponding mRNA sample.

24. out by magnetic force. The method according to claim 16, isolation of the magnetic beads is carried

25. A method of sequencing RNA, said method comprising the steps of:
providing a cDNA library comprising a plurality of sample-specific barcoded cDNAs, wherein said sample-specific barcoded cDNAs correspond to a unique mRNA sample defined by its unique barcode, wherein the contribution of each sample in the cDNA library is the same and wherein said sample-specific barcoded cDNAs are barcoded on their 5′ terminal end with a sequence which is specific to a unique sample;
amplifying said cDNAs from said library;
sequencing the amplification products.

26. The method according to claim 25, wherein the cDNA library is a library obtained by a method for the preparation of a cDNA library based many RNA samples comprising the steps of:
i) providing separately a plurality of mRNA samples;
ii) contacting separately each mRNA sample with biotinylated and barcoded oligo-dT sequences wherein the biotinylated and barcoded oligo-dT sequences are biotinylated at their 5′ end under annealing conditions to obtain, for each sample, sample-specific barcoded mRNA complexes;
iii) contacting separately for each sample, said sample-specific barcoded mRNA complexes with streptavidin Magnetic Beads at a pre-defined concentration, said pre-defined concentration being identical for all mRNA samples;
iv) incubating separately each sample with reverse transcription enzyme (RT) under reverse transcription reaction conditions;
v) isolating separately for each sample the magnetic beads from the reaction medium; and
vi) pooling together in a single set of samples all the isolated magnetic beads from each sample to obtain a cDNA library.

27. The method according to claim 25, wherein the amplifying step of cDNAs comprises at least one step selected from fragmentation or tagmentation, adapter ligation, DNA amplification, concentration measurement and DNA size distribution profiling.

28. A kit comprising:
biotinylated and barcoded deoxy-thymidine (oligo-dT) primers and
strepavidin magnetic beads or magnetic beads,
wherein said biotinylated and barcoded oligo-dT sequences comprise each:
a known sequence specific for each sample (barcode sequence);
a single strand sequence of deoxy-thymidine (dT) which is capable to anneal to any poly-A tail of mRNA molecules; and
a biotin group modification of 5′ end of the oligo-dT primer.

29. The kit according to claim 28, wherein strepavidin magnetic beads are optionally pre-functionalized with said barcoded oligo-dT primers.

30. The kit according to claim 28, wherein said deoxy-thymidine (oligo dT) sequences are selected from the sequences from SEQ ID NO: 23 to SEQ ID NO: 45.
